(19) United States
(12) Patent Application Publication
(10) Pub. No.: US 2024/0423736 A1
(43) Pub. Date: Dec. 26, 2024

(54) ACTIVE ROBOTIC SYSTEMS WITH USER CONTROLLER

(71) Applicant: Monogram Orthopedics Inc., Austin, TX (US)

(72) Inventors: Kamran SHAMAEI, Austin, TX (US); Sanath VURELLI, Round Rock, TX (US)

(73) Assignee: Monogram Orthopedics Inc., Austin, TX (US)

(21) Appl. No.: 18/781,074

(22) Filed: Jul. 23, 2024

Related U.S. Application Data
(63) Continuation of application No. PCT/US2023/061201, filed on Jan. 24, 2023.
(60) Provisional application No. 63/302,270, filed on Jan. 24, 2022.

(51) Int. Cl. A61B 34/37; A61B 17/00; A61B 17/15; A61B 34/00; A61B 34/10; A61B 34/32

(52) U.S. Cl. CPC A61B 34/37; A61B 17/15; A61B 34/25; A61B 34/32; A61B 34/74; A61B 2017/00973; A61B 2034/107

(57) ABSTRACT

Fully autonomous robotic systems and methods with user controls, interface and configurations. A robotic system includes an articulated arm, a cutting tool defining a cutting edge that cuts while being translated along a cutting pathway, an end effector coupled to an end arm segment of the articulated arm and the cutting tool and including a powered drive portion that translates the cutting tool along the cutting pathway, and an electronic controller. The robotic system is configured to autonomously adjust the relative orientation of arm segments of the articulated arm while the cutting tool translates along the cutting pathway to autonomously spatially translate the cutting tool and cut a material along at least one programmed cut path. The controller comprises a plurality of control inputs that a user can selectively actuate to control metrics of the movement of the cutting tool along the at least one programmed cut path.

### CROSS-REFERENCE TO RELATED APPLICATIONS

The present application is a bypass continuation of International Application No. PCT/US2023/061201, entitled Active Robotic Systems with User Controller, filed on Jan. 24, 2023, which perfects and claims priority benefit of U.S. Provisional Application No. 63/302,270, entitled Active Robotic Systems with User Controller, filed on Jan. 24, 2022, the entireties of which are hereby expressly incorporated herein by reference.

### FIELD OF THE DISCLOSURE

The following disclosure relates generally to improved surgical robot systems and related systems. More particularly, the following disclosure relates to autonomous surgical robotic and related methods that utilize an electronic controller to allow a user to control a cutting tool of the robot along determined cut paths without the user physically guiding the cutting tool.

### BACKGROUND

Powered cutting tools, such as oscillating cutting saws and rotary burrs, bitts or other cutting tools, have been used to reduce operating time and surgeon labor, and improve accuracy, in orthopedic surgical procedures. Such powered cutting tools enable faster and more accurate cutting of bone and other tissue during surgical procedures as compared to fully manual cutting tools, for example.

Recently, surgical robots have become available which can control the power cutting tools used in orthopedic surgical procedures so as to provide superior accuracy in cutting bone. Surgical robots include a robotic arm, which is typically articulated, that facilitates the gross movement of the cutting tool thereof, such as along cutting pathways.

The current state-of-the-art in orthopedic surgical robots are haptic robots that rely on a user to provide the gross movement of the cutting tool along a cut path (and typically a drive mechanism that operates the cutting tool along a cutting pathway along which the cutting tool is designed to cut). The orthopedic surgical robots are designed for user initiated cutting such that the robot itself is not actively or autonomously executing the cuts.

Such haptic current surgical robots are thus configured as hand-guided instruments that power the cutting tool and, at best, assist a user in translating the cutting tool to (and through) a patient, but require a user to manually move and direct the cutting tool along its cutting pathway (i.e., the robot is not actively executing the cuts). For example, some such haptic surgical robots include a handle and a trigger that a user manually utilizes to move and direct an active cutting tool along its cutting pathway. The user (e.g., a surgeon) must thereby manually provide inputs to the robot by physically moving it.

It is noted that some such haptic robotic cutting systems may electronically or autonomously define a cutting area/zone and a non-cutting area/zone, and actively prevent a user form translating the cutting tool into/through the non-cutting area to protect areas of a patient that should not be cut (e.g., accidently). However, such robotic systems do not actively or autonomously translate the cutting tool through the cutting area, as the user must physically move the cutting tool along the cut paths in the cutting area.

Haptic robotic cutting systems thereby require one or both of a user's hands to manually physically guide the cutting tool (and drive mechanism) to and through cut paths. In surgical applications, it is often necessary to surgically cut or resect a bone, cartilage and/or other tissue of a patient (e.g., a mammalian patient), such as during a surgical procedure. A surgeon or technician often does not have both of their hands readily available during surgical procedures, haptic robotic cutting systems are problematic. As haptic robotic cutting systems require user initiated cutting, they systems are relatively demanding on users (e.g., surgeons), limits a user's ability to perform other tasks (the suer loses use of at least one hand), and the robotic system often physically interfered with access to the patient. Still further, as current haptic robotic cutting systems require a user to physically define or control the cut paths, the location, quality, timing and pattern of cuts via the robotic system are reliant on the skill of the user.

Fully autonomous or active robotic systems and related methods that autonomously follow determined cut paths, without a user physically engaging and guiding the cutting tool, or drive mechanism, are desirable. For example, autonomous or active orthopedic and/or surgical robotic systems and related methods that autonomously follow determined cut paths would be advantageous to allow the user (e.g., surgeon) to perform other tasks.

The present disclosure provides improved robotic systems and related methods that autonomously/actively follow determined cut paths without a user physically engaging and guiding the cutting tool (or drive mechanism), but include an intuitive user controller for a limited amount of control of the cutting parameters or process by the user. The improved autonomous/active robotic systems provide for optimized efficient (potentially pre-planned) cut paths, allow the user to use both of their hands for other tasks, and free up working space about the robotic system, while providing a user with some level of control of the cutting parameters or process to ensure safe, proper, and efficient cuts.

While certain aspects of conventional technologies have been discussed to facilitate disclosure of Applicant's inventions, the Applicant in no way disclaims these technical aspects, and it is contemplated that the inventions may encompass one or more conventional technical aspects.

In this disclosure, where a document, act or item of knowledge is referred to or discussed, this reference or discussion is not an admission that the document, act or item of knowledge or any combination thereof was, at the priority date, publicly available, known to the public, part of common general knowledge, or otherwise constitutes prior art under the applicable statutory provisions; or is known to be relevant to an attempt to solve any problem with which this specification is concerned.

### SUMMARY

The present inventions may address one or more of the problems and deficiencies of current surgical robots, surgical robot system components and related surgical methods. However, it is contemplated that the inventions may prove useful in addressing other problems and deficiencies in a number of technical areas. Therefore, the claimed invention(s) should not necessarily be construed as limited to addressing any of the particular problems or deficiencies discussed herein.

The present disclosure is generally directed to surgical robots, surgical robotic systems and related surgical methods that include a controller that allows a user to electronically control parameters of movement of a cutting tool along a prescribed or determined cut path. The present disclosure provides configurations and methods of facilitating user inputs to an active or autonomous robotic system to control parameters of movement of a cutting tool along a prescribed or determined cut path. The surgical robotic systems and related surgical methods provide an electronic controller that provides intuitive user control of an active robot along a prescribed or determined cut path thereof.

In some embodiments, the surgical robotic systems are configured such that the electronic controller provides for selective advancement and/or retracement retracement/retraction of the cutting tool along a prescribed or determined cut path. In some embodiments, the surgical robotic systems are configured such that the electronic controller provides for selective speeding up and/or slowing down the velocity of the cutting tool along a prescribed or determined cut path. The surgical robotic systems are thereby configured such that a user has limited control over the parameters of movement of the cutting tool along a prescribed or determined cut path to ensure the cutting tool properly or appropriately cuts a material (e.g., bone) according to the prescribed or determined cut path. For example, it may be desirable and/or advantageous to advance and retract the cutting tool along a programmed cut path, and/or speed up or slow down the velocity of the movement of the cutting tool along a programmed cut path, at critical moments, such as at initial contact with the cutting surface to ensure, facilitate or encourage engagement of the cutting tool with the workpiece without deflection thereof to initiate a cut along the intended programmed cut path.

In some embodiments, the electronic controller of the surgical robotic systems of the present disclosure is configured as a hands-free device such that it is configured to be utilized via a foot of a user. In some other embodiments, the electronic controller of the surgical robotic systems of the present disclosure is a hand-held device such that it is configured to be utilized via a single hand of a user. The electronic controller of the surgical robotic systems is configured to take inputs from a user to command the robot along a determined, prescribed or programmed cut path of the cutting tool. The electronic controller may feature tactile control inputs that a user can physically engage or activate to correspondingly control a parameter of the movement of the cutting tool along a determined, prescribed or programmed cut path of the cutting tool that is being currently executed. In some embodiments, the tactile control inputs may comprise electrical switches, which may provide tactile or physical feedback/movement. In some such embodiments, the electrical switches are configured as depressible buttons, rocker switches, a foot pedal or a combination thereof.

It is noted that the cutting tool may be any cutting tool, such as but not limited to a surgical cutting tool configured to cut or resect tissue. In one exemplary embodiment, the cutting tool is an oscillating cutting blade or saw (e.g., a sagittal surgical saw blade). In another exemplary embodiment, the cutting tool is a rotary cutting tool, bit or blade. Similarly, the end effector may be any instrument configured to move (e.g., reciprocate or rotate) the cutting tool along a cutting direction or pathway that the cutting edge is configured to cut when moved there-along, such as a powered sagittal saw end effector or a powered rotary end effector. The end effector may be coupled between a distal arm segment of an articulated robotic arm of the robot and the cutting tool, and thereby may be translated on the same path as the cutting tool. While the end effector may translate the cutting tool along its cutting pathway, the articulated arm may provide the gross movement of the cutting tool (at least partially) to and through a material along a prescribed cutting path to cut the material.

In one aspect, the present disclosure provides a robotic system comprising: an articulated arm comprising a plurality of arm segments defining longitudinal axes, and adjustable joints coupled between adjacent arm segments that are configured to adjust the orientation of the axes of the adjacent arm segments; a cutting tool defining a cutting edge that is configured to cut while being translated along a cutting pathway; an end effector coupled to an end arm segment of the plurality of arm segments and the cutting tool, the end effector comprising a powered drive portion that translates the cutting tool along the cutting pathway; and an electronic controller. The robotic system is configured to autonomously adjust the relative orientation of the arm segments of the articulated arm while the cutting tool translates along the cutting pathway to autonomously spatially translate the cutting tool and cut a material along at least one programmed cut path. The controller comprises a plurality of control inputs that a user can selectively actuate to control the movement of the cutting tool while executing the at least one programmed cut path.

In some embodiments, the plurality of control inputs comprises a first control input that, when selectively actuated by a user, is configured to direct the robotic system to advance the cutting tool along a current programmed cut path that the robotic system is executing. In some embodiments, the plurality of control inputs comprises a second control input that, when selectively actuated by a user, is configured to direct the robotic system to retreat the cutting tool along a current programmed cut path that the robotic system executed immediately previous thereto.

In some embodiments, the plurality of control inputs comprises a third control input that, when selectively actuated by a user, is configured to direct the robotic system to translate the cutting tool along a current programmed cut path that the robotic system is executing at an increased velocity as compared to a current programmed velocity. In some embodiments, the plurality of control inputs comprises a fourth control input that, when selectively actuated by a user, is configured to direct the robotic system to translate the cutting tool along a current programmed cut path that the robotic system is executing at a decreased velocity as compared to a current programmed velocity.

In some embodiments, the plurality of control inputs comprises a fifth control input that, when selectively actuated by a user, is configured to stop the end effector from translating the cutting tool along the cutting pathway. In some embodiments, the plurality of control inputs comprises a sixth control input that, when selectively actuated by a user, is configured to register a current point of the cutting tool along the cutting pathway that the robotic system is executing.

In some embodiments, the plurality of control inputs comprise a plurality of electrical switches. In some embodiments, the robotic system is operated by a computer system.

In some embodiments, the controller is configured as a foot controller such that the plurality of control inputs are configured to be selectively actuated by a user's foot. In some embodiments, the foot controller comprises a foot pedal that is configured to be selectively engaged by the underside of a user's foot and a housing positioned adjacent to at least one side of the foot pedal, and wherein the foot pedal is configured such that the user can articulate the foot pedal between a first forefoot position with a forefoot portion of the foot pedal being depressed, and a second hindfoot position with a hindfoot portion of the foot pedal being depressed. In some embodiments, the first forefoot position of the foot pedal actuates a first control input that is configured to direct the robotic system to advance the cutting tool along a current programmed cut path that the robotic system is executing when selectively actuated by a user.

In some embodiments, the second hindfoot position of the foot pedal actuates a second control input second control input that is configured to direct the robotic system to retreat the cutting tool along a current programmed cut path that the robotic system executed immediately previous thereto when selectively actuated by a user. In some embodiments, the housing comprises a plurality of the plurality of control inputs that are configured to be selectively engaged by the user's foot. In some embodiments, the housing comprises a third control input that, when selectively actuated by a user, is configured to direct the robotic system to translate the cutting tool along a current programmed cut path that the robotic system is executing at an increased velocity as compared to a current programmed velocity. In some embodiments, the housing comprises a fourth control input that, when selectively actuated by a user, is configured to direct the robotic system to translate the cutting tool along a current programmed cut path that the robotic system is executing at a decreased velocity as compared to a current programmed velocity. In some embodiments, the third and fourth control inputs are positioned on opposing lateral sides of the foot pedal. In some embodiments, the housing comprises a fifth control input that, when selectively actuated by a user, is configured to stop the end effector from translating the cutting tool along the cutting pathway. In some embodiments, the foot pedal is configured to be selectively rotated along a medial-lateral direction by a user's foot, and wherein medial-lateral rotation of the foot pedal is configured to activate a sixth control input that, when selectively actuated by a user, is configured to register a current point of the cutting tool along the cutting pathway that the robotic system is executing.

In some embodiments, the controller is configured as a handheld controller such that the plurality of control inputs are configured to be selectively actuated by one or more fingers of a hand of the user. In some embodiments, the handheld controller comprises a grip portion configured to be grasped by a user's palm and one more fingers of the user's hand, and a head portion comprising a plurality of the plurality of control inputs on an upper side thereof.

In some embodiments, the plurality of control inputs on the head portion comprise at least one of: a first control input that, when selectively actuated by a user, is configured to direct the robotic system to advance the cutting tool along a current programmed cut path that the robotic system is executing; a second control input that, when selectively actuated by a user, is configured to direct the robotic system to retreat the cutting tool along a current programmed cut path that the robotic system executed immediately previous thereto; a third control input that, when selectively actuated by a user, is configured to direct the robotic system to translate the cutting tool along a current programmed cut path that the robotic system is executing at an increased velocity as compared to a current programmed velocity; a fourth control input that, when selectively actuated by a user, is configured to direct the robotic system to translate the cutting tool along a current programmed cut path that the robotic system is executing at a decreased velocity as compared to a current programmed velocity; and a fifth control input that, when selectively actuated by a user, is configured to register a current point of the cutting tool along the cutting pathway that the robotic system is executing.

In some embodiments, the cutting tool is a rotary cutting tool, and wherein the rotary cutting tool is configured to cut when translated longitudinally and rotated about a longitudinal an axis thereof. In some embodiments, the cutting tool is a sagittal cutting blade with cutting teeth positioned at a longitudinal end thereof, the sagittal cutting blade being configured to cut when translated longitudinally and oscillated along the cutting pathway that lies in a plane in which the blade is aligned about an axis of oscillation. In some embodiments, the cutting tool is configured to cut bone, and wherein the at least one programmed cut path extends through a first bone of a patient such that the cutting tool cuts the first bone when executing the least one programmed cut path.

In another aspect, the present disclosure provides a method of cutting a material comprising utilizing a robotic system as described above to translate the cutting tool along the cutting pathway and the least one programmed cut path to cut the material.

In some embodiments, the material comprises a bone of a mammalian patient, and wherein the cutting tool comprises a rotary cutting tool or a sagittal cutting blade.

It should be appreciated that all combinations of the foregoing aspects and additional concepts discussed in greater detail below (provided such concepts are not mutually inconsistent) are contemplated as being part of the inventive subject matter and to achieve the advantages disclosed herein.

These and other objects, features and advantages of this disclosure will become apparent from the following detailed description of the various aspects of the disclosure taken in conjunction with the accompanying drawings.

### BRIEF DESCRIPTION OF THE DRAWINGS

These and other features, aspects, and advantages of the present disclosure will become better understood when the following detailed description is read with reference to the accompanying drawings, which are not necessarily drawn to scale and in which like reference numerals represent like aspects throughout the drawings, wherein:

FIG. 1 illustrates, in one example, a robotic system with a rotary cutting tool, in accordance with one or more aspects of the present disclosure.

FIG. 2 illustrates, in another example, a robotic system with an oscillating cutting blade, in accordance with one or more aspects of the present disclosure.

FIG. 3 illustrates, in one example, an elevational perspective view of an end arm segment, an end effector and the oscillating cutting blade of the robotic system of FIG. 2, in accordance with one or more aspects of the present disclosure.

FIG. 4 illustrates, in one example, an elevational perspective view of a series of determined cut paths of a cutting tool of a surgical robotic system extending along/though a bone, in accordance with one or more aspects of the present disclosure.

FIG. 5 illustrates, in one example, an elevational perspective view of an active robotic system foot activated controller, in accordance with one or more aspects of the present disclosure.

FIG. 6 illustrates, in one example, another elevational perspective view of the active robotic system foot activated controller of FIG. 4, in accordance with one or more aspects of the present disclosure.

FIG. 7 illustrates, in one example, a top view of the active robotic system foot activated controller of FIG. 4, in accordance with one or more aspects of the present disclosure.

FIG. 8 illustrates, in one example, an elevational perspective view of an active robotic system hand activated controller, in accordance with one or more aspects of the present disclosure.

FIG. 9 illustrates an elevational perspective view of the robotic system of FIG. 2, in accordance with one or more aspects of the present disclosure.

FIG. 10 illustrates side view of a workspace with a patient workpiece and the robotic system of FIG. 2 with vertically oriented an angled articulating arms, in accordance with one or more aspects of the present disclosure.

FIG. 11 illustrates an elevational perspective view of a robotic system with a cart mounted autonomous/active surgical cutting robot and a separate navigation or guidance cart, in accordance with one or more aspects of the present disclosure.

FIG. 12 illustrates an elevational perspective view of the robotic system of FIG. 11 performing a cutting operation on a patient/workpiece, in accordance with one or more aspects of the present disclosure.

FIG. 13 illustrates another elevational perspective view of the robotic system of FIG. 11 performing a cutting operation on a patient/workpiece, in accordance with one or more aspects of the present disclosure.

FIG. 14 illustrates another elevational perspective view of the robotic system of FIG. 11 performing a cutting operation on a patient/workpiece, in accordance with one or more aspects of the present disclosure.

FIG. 15 illustrates a top plan view of the robotic system of FIG. 11 performing a cutting operation on a patient/workpiece, in accordance with one or more aspects of the present disclosure.

FIG. 16 illustrates a side view of the robotic system of FIG. 11 performing a cutting operation on a patient/workpiece, in accordance with one or more aspects of the present disclosure.

FIG. 17 illustrates, in one example, a graphic representation of a computer system and associated devices to incorporate and/or use aspects described herein, in accordance with one or more aspects of the present disclosure.

### DETAILED DESCRIPTION

Aspects of the present disclosure and certain examples, features, advantages, and details thereof, are explained more fully below with reference to the non-limiting examples illustrated in the accompanying drawings. Descriptions of well-known materials, fabrication tools, processing techniques, etc., are omitted so as not to unnecessarily obscure the relevant details. It should be understood, however, that the detailed description and the specific examples, while indicating aspects of the disclosure, are given by way of illustration only, and are not by way of limitation. Various substitutions, modifications, additions, and/or arrangements, within the spirit and/or scope of the underlying inventive concepts will be apparent to those skilled in the art from this disclosure.

Approximating language, as used herein throughout disclosure, may be applied to modify any quantitative representation that could permissibly vary without resulting in a change in the basic function to which it is related. Accordingly, a value modified by a term or terms, such as “about” or “substantially,” is not limited to the precise value specified. For example, these terms can refer to less than or equal to +5%, such as less than or equal to +2%, such as less than or equal to +1%, such as less than or equal to +0.5%, such as less than or equal to +0.2%, such as less than or equal to +0.1%, such as less than or equal to +0.05%. In some instances, the approximating language may correspond to the precision of an instrument for measuring the value. Any examples of operating or configuration parameters are not exclusive of other parameters of the disclosed embodiments.

Terminology used herein is for the purpose of describing particular examples only and is not intended to be limiting. As used herein, the singular forms “a”, “an” and “the” are intended to include the plural forms as well, unless the context clearly indicates otherwise. Furthermore, references to “one example” are not intended to be interpreted as excluding the existence of additional examples that also incorporate the recited features. Moreover, unless explicitly stated to the contrary, the terms “comprising” (and any form of “comprise,” such as “comprises” and “comprising”), “have” (and any form of “have,” such as “has” and “having”), “include” (and any form of “include,” such as “includes” and “including”), and “contain” (and any form of “contain,” such as “contains” and “containing”) are used as open-ended linking verbs. As a result, any examples that “comprises,” “has,” “includes” or “contains” one or more step or element possesses such one or more step or element, but is not limited to possessing only such one or more step or element.

As used herein, the terms “may” and “may be” indicate a possibility of an occurrence within a set of circumstances; a possession of a specified property, characteristic or function; and/or qualify another verb by expressing one or more of an ability, capability, or possibility associated with the qualified verb. Accordingly, usage of “may” and “may be” indicates that a modified term is apparently appropriate, capable, or suitable for an indicated capacity, function, or usage, while taking into account that in some circumstances the modified term may sometimes not be appropriate, capable or suitable. For example, in some circumstances, an event or capacity can be expected, while in other circumstances the event or capacity cannot occur—this distinction is captured by the terms “may” and “may be.”

The term “coupled” and like terms are used herein to refer to both direct and indirect connections. As used herein and unless otherwise indicated, the term “entirety” (and any other form of “entire”) means at least a substantial portion, such as at least 95% or at least 99%. The term “entirety” (and any other form of “entire”), as used herein, is thereby not limited to 100%, unless otherwise indicated. As used herein, the term “layer”

Components, aspects, features, configurations, arrangements, uses and the like described, illustrated or otherwise disclosed herein with respect to any particular embodiment may similarly be applied to any other embodiment disclosed herein.

As described further below, surgical robots, surgical robotic systems and related surgical methods that include a controller that allows a user to electronically control parameters of movement of a cutting tool along a prescribed or determined cut path are disclosed. The surgical robotic systems and methods provide for user inputs to an active or autonomous robotic system to control parameters of movement of a cutting tool along a prescribed or determined cut path. The surgical robotic systems and related surgical methods comprise an electronic controller that provides intuitive user control of an active robot along a prescribed or determined cut path thereof.

In some embodiments, the surgical robotic systems are configured such that the electronic controller provides for selective advancement and/or retracement retracement/retraction of the cutting tool along a prescribed or determined cut path. In some embodiments, the surgical robotic systems are configured such that the electronic controller provides for selective velocity adjustment (e.g., speeding up and/or slowing down) of the cutting tool along a prescribed or determined cut path.

The surgical robotic systems provide a user with limited selective control over the parameters of movement of the cutting tool along a prescribed or determined cut path, rather than the configuration of the cut path itself, to ensure the cutting tool properly or appropriately cuts a material (e.g., bone) according to the prescribed or determined cut path. For example, the controller may be configured to provide user control over selective advancement and retraction of the cutting tool along a programmed cut path, and/or adjust (e.g., increase and/or decrease) the velocity of the movement of the cutting tool along the programmed cut path. Selective user control over movement parameters of the cutting tool when it is executing a cut along a programmed cut path may be particularly beneficial at critical moments of a cutting operation. For example, such selective user control over movement parameters of the cutting tool may be advantageous at initial contact of the cutting tool with the cutting surface to ensure, facilitate or encourage engagement of the cutting tool with the workpiece, without deflection thereof, to properly initiate a cut along the intended programmed cut path.

In some embodiments, the electronic controller of the surgical robotic systems is configured as a hands-free device such that it is configured to be utilized via a foot of a user. In some other embodiments, the electronic controller of the surgical robotic systems of the present disclosure is a hand-held device such that it is configured to be utilized via a single hand of a user.

The electronic controller of the surgical robotic systems is configured to allow a user to provide or enter inputs from a user that command the robot to alter parameters of movement of the cutting tool along a determined, prescribed or programmed cut path. The electronic controller may feature tactile control inputs that a user can physically engage or activate to correspondingly control a parameter of the movement of the cutting tool along a determined, prescribed or programmed cut path of the cutting tool that is currently being executed by the robotic system. In some embodiments, the tactile control inputs may comprise electrical switches, which may provide tactile or physical feedback/movement. In some such embodiments, the electrical switches are configured as depressible buttons, rocker switches, a foot pedal or a combination thereof.

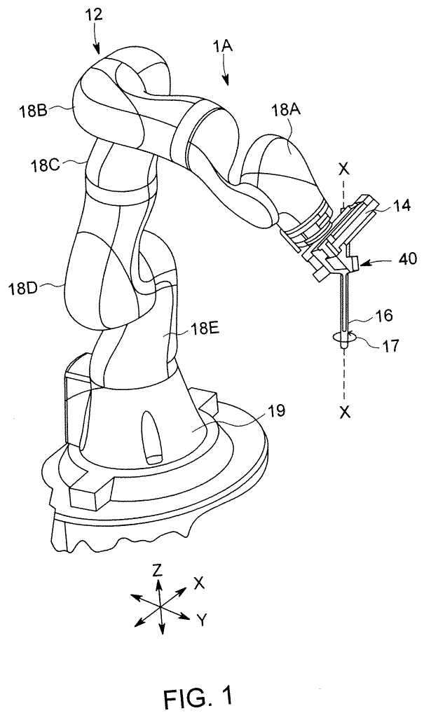

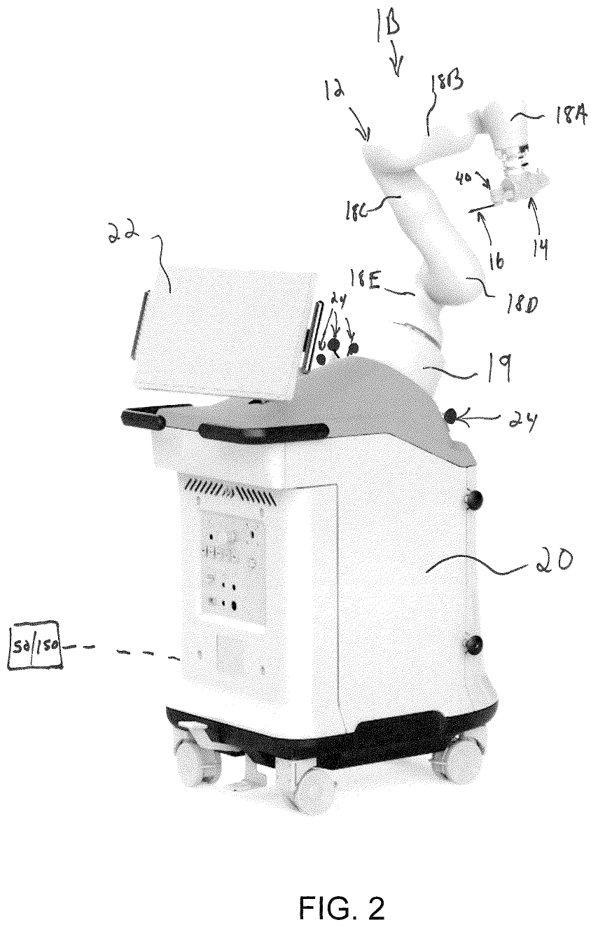

As shown in FIGS. 1 and 2, a robot or robotic system 1A, 1B according to the present disclosure may include, inter alia, an articulated robotic arm 12, an end effector 14 and a cutting tool or device 16. As depicted in FIGS. 1 and 2, a robotic system 1A, 1B may be configured as a surgical robot. For example, the surgical robot 1A, 1B may be biocompatible, and configured be sterilized to such a degree, as required in surgical settings. However, in other embodiments, the robot 1A, 1B may be configured as an industrial or other non-surgical robotic devices or systems. The term “surgical robot” as used herein in reference to the exemplary illustrative robot/robotic system embodiments, and is not meant in a limiting sense, and any and all description herein directed to a “surgical robot” or the like equally applies to a generic robot/robotic system or an industrial or other non-surgical robot/robotic system.

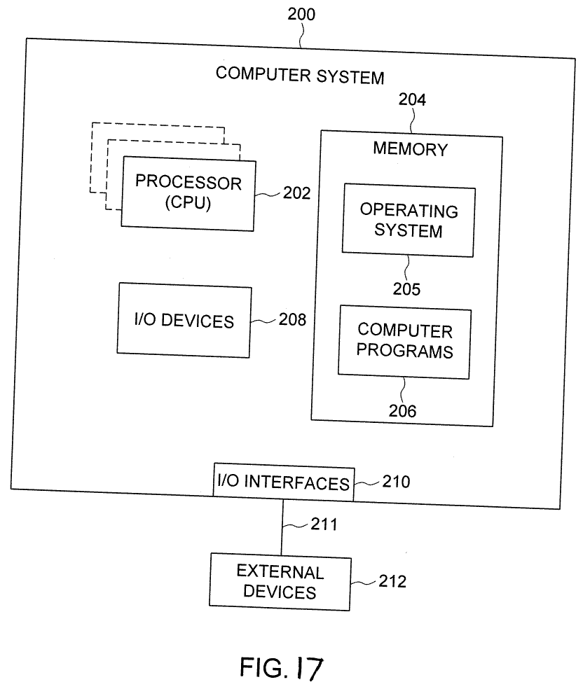

The robotic system 1A, 1B may be operably connected to a computer system (e.g., memory, processor, etc.), as shown in FIG. 17 for example, that controls movement of the cutting tool 16 along one or more determined, prescribed or programmed cut paths, via movement of the articulated arm 12 for example, and potentially operation of the end effector 14. For example, in some embodiments, the robotic system 1A, 1B may comprise a control unit with programming, and potentially a user interface (UI) 22 a shown in FIG. 2. The control unit may include at least one processing circuit, at least one input/output device, and at least one storage device or memory having at least one database or cutting instructions stored therein. The control unit may have a control algorithm or programming code for controlling the position of the cutting tool 16 (such as via the joint angle between segments of the articulated arm 12, for example). The control algorithm or programming code may be a default control algorithm or include inputs from, for example, the UI and/or another interface.

As shown in FIGS. 1 and 2, the articulated arm 12 may extend from a base 19 and include a plurality of rigid arm or body segments/parts 18A, 18B, . . . 18N, and a plurality of joints that connect adjacent segments (and a first or base segment to the base). The plurality of joints may include, for example, four, five or six individual segments that are coupled together via three, four or five joints, respectively. In some other embodiments, the articulated arm 12 may include at least two segments and at least one joint coupling the at least two segments together, or more than six segments and more than five joints coupling the segments together.

Each arm segment of the articulated arm 12 may define an axial axis extending along its longitudinal length. The joints may be configured such that the arm segments can rotate about their axes and/or articulate angularly with respect to each other such that the axes of adjacent segments are angularly offset. In some embodiments, one or more of the joints may be configured to allow multiple degrees of freedom between adjacent arm segments (and, potentially, the base segment and the base). In some such embodiments, at least one of the joints may be configured to provide six degrees of freedom.

The articulated arm 12 may further comprise motors, actuators or other adjustment devices that are configured to adjust the axial rotation and/or angular orientation between adjacent segments. In this way, the robotic system 1A, 1B can control the arrangement of the articulated arm 12 to translate the cutting tool 16 three-dimensionally in space and relative to a workpiece (e.g., a patient) to, ultimately, cut one or more portions of the workpiece. As noted above, the robotic system 1A, 1B may include control software that dictates or instructs, inter alai, the articulated arm 12 of the robotic system 1A, 1B to adjust in particular ways (i.e., adjustment of the joints) to accomplish determined, prescribed or programmed cut path movements of the cutting tool 16. Stated differently, the robotic system 1A, 1B may include control software that dictates or instructs, inter alai, the articulated arm 12 of the robotic system 1A, 1B to adjust in particular ways (i.e., adjustment of the joints) to translate the cutting tool 16 in three-dimensional space along one or more (e.g., a series of a plurality of) determined, prescribed or programmed cut paths of the cutting tool 16 to cut the workpiece along or according to the determined, prescribed or programmed cut paths. The paths of the cutting tool 16 may thereby be predetermined, pre-prescribed or pre-programmed in the robotic system 1A, 1B such that the user does not dictate the cut paths of the cutting tool 16 that the cutting tool 16 travels along to cut the workpiece. As explained further below, the user may be provided with control over parameters or metrics of the movement of the cutting tool 16 along the cut paths via a controller, but not the configuration or three-dimensional parameters of the cut paths themselves.

The base 19 of the surgical robotic system 1A, 1B may be fixed to, for example, a movable cart 20 a shown in FIG. 2, or to the ground or base structure as shown in FIG. 1, such that the base may provide a fixed frame of reference for defining the position, orientation, and motion of the plurality of joints and the plurality of arm segments 18A, 18B, . . . 18N relative to the base 19. The base 19, or base structure or cart 20, may be used to define a frame of reference, such as, for example, a set of three-dimensional axes (e.g., x, y, z), which may be used to define positions, orientations, and motions of the surgical robotic system 1A, 1B and of objects relative to the surgical robotic system 1A, 1B. For example, as shown in FIG. 1, the robotic system 1A, 1B may include an array of fiducials 24 of a known relative position and orientation which a camera or other detection system of the robotic system 1A, 1B can utilize to determine the position and/or orientation of the robotic system 1A, 1, such as the base 19 and/or the first arm segment (e.g., arm segment 18E) thereof. It is notes that the camera or other detection system of the robotic system 1A, 1B may also determine the position and/or orientation of the cutting tool 16 (e.g., the tip thereof) directly/independently and/or based on the position and/or orientation and the configuration of the arm 12. A frame of reference defined relative to the base 19 may also be known as a world frame, a base, a base frame, a frame, or a tool frame. It is noted that with the position and orientation of an object defined or calculated in relation to the fixed frame of reference, the object may also be defined in the same frame of reference as the robotic system 1A, 1B, and the surgical robotic system 1A, 1B may calculate the position and orientation of the object. As such, the surgical robotic system 1A, 1B may programmably interact with the defined objects, positions, and/or orientations.

As shown in FIGS. 1 and 2, the robotic system 1A, 1B may include an end effector 14 coupled (e.g., rotatably coupled) to an end, last or termination arm segment 18 of the articulated arm 12, such as via a rotatable connector assembly therebetween. The rotatable connector assembly 19 may be configured such that the end effector 14 is rotatable about a longitudinal axis of the end arm segment 18. As shown in FIGS. 1-3, the end effector 14 may be configured such that the axis of the end arm segment 18 and the axis X-X of the cutting tool 16, along which the cutting direction of the cutting tool 16 extends, are angled with respect to each other. In the illustrated exemplary embodiment, the axis X-X of the cutting tool 16 (i.e., the direction the cutting tool 16 is configured to cut) and the axis of the end arm segment 18 are oriented perpendicular (or normal) to each other.

Referring further to FIGS. 1 and 2, since the position, orientation, and motion of the plurality of joints and the plurality of arm segments relative to the base may be defined, and the angular orientation of the end effector 14 with respect to the end arm segment 18 of the articulated arm 12, the position and orientation of the cutting tool 16 extending from the end effector 14 can be calculated or determined by the robotic system 10, or vice versa. In another embodiment, the fiducial array 24 may be utilized to determine/detect the position and/or orientation of the system 1A, 1B (e.g., the base 19) via a camera or other detection system, and/or the position and orientation of the cutting tool 16 (e.g., a cutting portion or tip thereof) may be determined/detected via the camera or other detection system. It is understood that the exemplary illustrative cutting tool 16 is configured for cutting bone or other tissue, however the cutting tool 16 may be replaceable with a different cutting tool or a non-cutting implements that may function as, for example, a marking device or a viewing device.

As shown in FIGS. 1-3, the cutting tool 16 of the exemplary robotic systems 1A, 1B may include an attachment, tang or hub portion at a proximal end portion that couples with an attachment mechanism 40 of the end effector 14. For example, the end effector 14 may include a chuck or other attachment mechanism 40 configured to mate with the attachment portion of the cutting tool 16, and removably secure the cutting tool 16 and the end effector 14 together. The end effector 14 may thereby be capable of physically translating or moving the cutting tool 16 along the cutting pathway 17 along which the cutting tool 17 is configured to cut. The axis X-X of the cutting tool 16 may extend through the attachment portion 40 and the end effector 14, and the cutting blade 16 (and the end effector 14) may be longitudinally extended along the axis X-X.

As shown in FIG. 1, in some embodiments, an exemplary robotic system 1A comprises a cutting tool 16 configured as an axially extending rotary cutting tool. The cutting tool 16 of the exemplary robotic system 1A is configured to cut along a cutting pathway 17 that extends about the longitudinal axis X-X of the cutting tool 16 (i.e., is configured to cut when the cutting tool 16 is rotated about its axis X-X and translate axially/longitudinally through a workpiece). The end effector 14 may thereby be configured to provide such rotation or torque to the cutting tool 16 such that it is rotated along the cutting pathway 17.

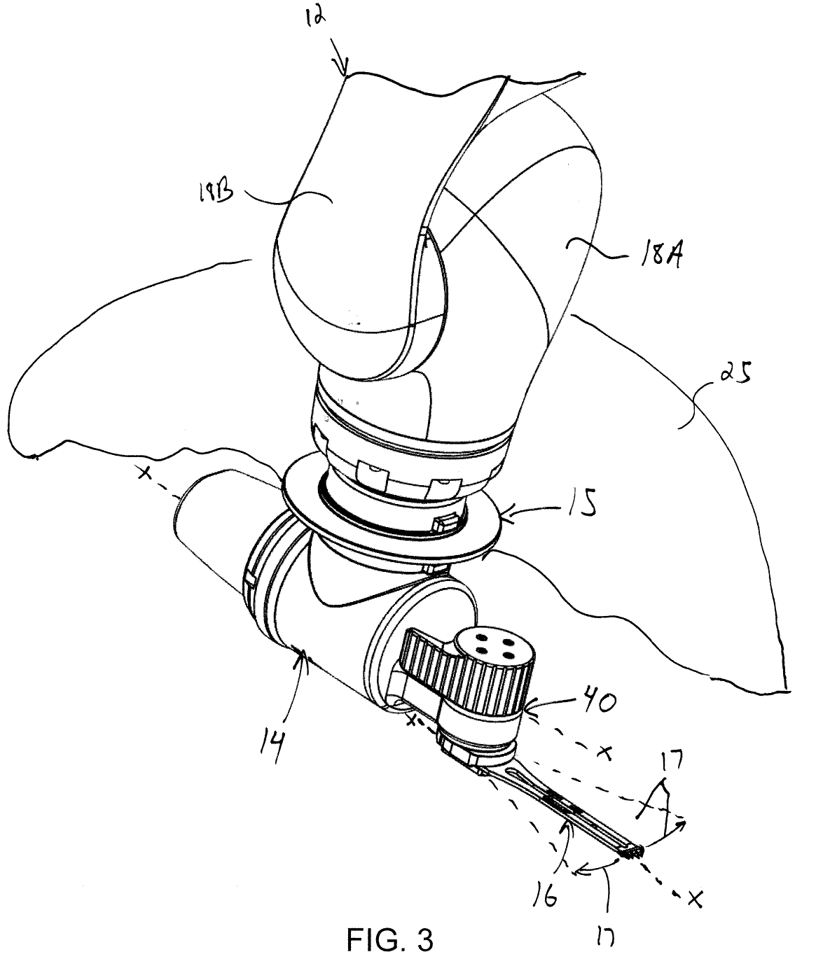

As shown in FIGS. 2 and 3, in some other embodiments, an exemplary robotic system 1B comprises a cutting tool 16 configured as a cutting blade. For example, as shown in FIGS. 2 and 3, the cutting tool 16 of the exemplary robotic system 1B may be a saw blade that has a thin, flat, elongated shape with a cutting edge at a distal tip or end portion of a blade body portion. The thin, flat design may minimize the size of the blade's kerf and allow the blade to make an accurate, straight cut. The cutting edge may be generally oriented along a direction that is orthogonal to the direction of blade elongation and contains a plurality of teeth and/or abrasives. Thus, when the blade 16 is translated along a cut pathway 17, the cutting edge can be translated axially/longitudinally against and through a workpiece (e.g., a bone or other tissue that requires resection) as it is translated along its cutting pathway or direction 17.

The cutting blade 16 (e.g., at least the cutting edge thereof) of the exemplary robotic system 1B may be configured to cut when moved/translated in the cutting pathway 17, such as in a reciprocating motion (along forward and/or back strokes), along a linear direction (colinear with the cutting edge), along a plane (e.g., two dimensions) or in a three-dimensional pattern. The exemplary illustrative cutting blade 16 is configured to be pivoted back and forth, or oscillated, in the cutting pathway 17 which extends along the plane in which the cutting blade 16 is oriented and is orthogonal to the direction of blade elongation. The cutting blade 16 of the exemplary robotic system 1B may be designed such that the cutting direction or pathway 17 oscillates linearly laterally or in an arc extended along the plane of the blade 16. The blade 16 may thereby be configured as a sagittal saw blade.

With reference to FIG. 3, in some embodiments, the end effector 14 of the robotic system 1B may be configured to oscillate (i.e., translate in a back and forth manner) the cutting tool 16, which is configured as a cutting blade, along the oscillatory cutting direction or pathway 17. As explained above, the cutting tool 16 may be configured as a sagittal cutting blade, and the oscillatory cutting pathway 17 may extend along a plane defined by the blade. When the cutting tool is configured as a sagittal saw blade, the blade performs a cutting action by being translated in a cut path extending along the longitudinal axis X-X (e.g., via the articulated arm 12, at least in part) (i.e., longitudinally/axially) in a direction extending from the coupling portion to the tip portion thereof as the blade (and the cutting teeth thereof) is being oscillated along the oscillatory cutting pathway 17 (i.e., in cutting strokes). Because of the motion along the oscillatory cutting pathway 17 applied by the end effector 14, and the forward pressure applied by the robotic system 1B (e.g., via the articulated arm 12, at least in part) along a cut path, the teeth of the cutting blade 16 cut and separate material of a workpiece (e.g., tissue, such as bone tissue).

As shown in FIG. 2, in some embodiments, the base portion 19 coupled to the first segment 18E of the articulating arm 12 may be coupled or mounted on a movable cart 20. The cart 20 may thereby provide a movable base for the robotic system 1B to make the robotic system 1B a compact transportable or movable cutting robot construct, as shown in FIG. 2. The cart 20 may also include a screen or graphical user interface 22 that allows user to see and/or visualize the operation of the robotic system 1B. For example, the robotic system 1B may be configured to display the status of the operation of the cutting tool 16 (e.g., via the operation of the end effector 14), the position and/or orientation of the cutting tool 16 (potentially relative to a patient or other workpiece), and/or the status of one or more cut paths or cutting operations. The screen or graphical user interface 22 may also allow a user to operate the robotic system 1B (e.g., alter a metric or characteristic of the operation of the robotic system 1B). In some embodiments, as shown in FIG. 2, the base 19 of the articulating arm 12, and thereby at least the base portion of the articulating arm 12, may be positioned on one end or side of the cart 22, and the screen may be positioned on an opposing end or side of the cart 2. As explained further below, the base portion 19 may be angled with respect to vertical such that the articulating arm 12 is biased off the respective side or end of the cart 22 and away from a user at the end or side of the cart 20 with the screen or graphical user interface 22. In some embodiments, as shown in FIG. 2, the robotic system 1B also includes one or more controller 50, 150 that is configured to operate the robotic system 1, which may be wired or wireless connected to the cart 20 for example.

As shown in FIG. 3, the robotic system 1B may include a connector assembly 15 that coupled the end effector 14 and a rotatable joint at the end of the end or last arm segment 18A together. In some embodiments, the connector assembly 15 is configured such that a user can manually selectively couple and decouple the end effector 14 to the arm 12. In some such embodiments, the connector assembly 15 may be configured as a quick connector. In one exemplary embodiment, the connector assembly 15 comprises male and female connector portions or components that removably couple together to selectively fix the end of the end or last arm segment 18A to couple them together.

The connector assembly 15 may comprise one or more connectors or connector portions. For example, in some embodiments the connector assembly may include an intermediate flange portion that couples to the side of the end effector 14 and the joint at the end of the end or last arm segment 18A to couple them together, as shown in FIG. 3. In some such embodiments, a first side of the flange portion may include a first connector and the side of the end effector 14 may include a second connector that mates with the first connector to removably fixedly couple the flange portion and the end effector 14 together. In some such embodiments, a second side of the flange portion may include a third connector and the joint at the end of the end or last arm segment 18A may include a fourth connector that mates with the third connector to removably fixedly couple the flange portion and the last arm segment 18A together. In some such embodiments, the first and fourth connectors may be configured male connectors, and the second and third connectors may be configured as female connectors.

As shown in FIGS. 3 and 16, in some embodiments, the robotic system 1B may include a surgical drape or curtain 25 coupled to the flange portion of the connector assembly 15 between the end effector 14 and the end segment 18A of the articulating arm 12. The surgical drape or curtain 25 (and the flange portion) may extend fully about the connector assembly 15 and otherwise be configured to form a sterility barrier. As the surgical drape or curtain 25 is positioned between the arm 12 (and the rest of the robotic system 1B) and the end effector 14, the surgical drape or curtain 25 can be effective in creating a sterility barrier or seal between the end effector 14 (and the attachment mechanism 40 and the cutting tool 16 attached thereto) and patient 75, and the other portions of the robotic system 1, as shown in FIG. 16. In this way, the end effector 14, attachment mechanism 40 and/or the cutting tool 16 can be changed or adjusted within the sterile area proximate to the patient without having to remove or otherwise break the sterility barrier.

As shown in FIG. 4, the robotic system 1A, 1B may include a plurality of determined, prescribed or programmed cut paths 32 of the cutting tool 16 to effectuate cut of a workpiece 30 along or according to the determined, prescribed or programmed cut paths 32. The robotic system 1A, 1B may include control software that dictates or instructs, inter alai, the articulated arm 12 of the robotic system 1A, 1B to adjust in particular ways (i.e., adjustment of the joints) to translate the cutting tool 16 in three-dimensional space along the determined, prescribed or programmed cut paths 32 of the cutting tool 16 to cut the workpiece 30 along or according to the determined, prescribed or programmed cut paths 32. It is noted that the end effector 14 translates the cutting tool 16 along its cutting pathway 17 while the robotic system 1A, 1B is translating the cutting tool 16 (via the articulated arm, at least partially) along the determined, prescribed or programmed cut paths 32 (which the user does not determine or effectuate, but rather are fully autonomously determined and/or performed by the active the robotic system 1A, 1B).

The cut paths 32 of the cutting tool 16 may thereby be predetermined, pre-prescribed or pre-programmed in/via the robotic system 1A, 1B such that the user does not dictate the cut paths 32 of the cutting tool 16 that the cutting tool 16 travels along to cut the workpiece 30. As explained further below, the user may be provided with control over parameters or metrics of the cutting tool 16 as it moves along the cut paths 32 via a controller, but not the configuration or three-dimensional parameters of the cut paths 32 themselves (i.e., the user does not determine or control the portions of the workpiece (i.e., bone) that are removed/cut (i.e., the cut paths themselves). It is noted that the cut paths 32 may thereby include entry positions where the cutting tool 16 first initiates cutting of the workpiece 30 and enters the workpiece 30, as well as three-dimensional spatial pathways extending from the entry positions through the workpiece 30, and represent removed or cut portions of the workpiece 30.

As shown in FIG. 4, in some embodiments, the cut paths 32 comprise spatial pathways to and through a (bone) workpiece 30. The cut paths 32 may thereby be configured to resect or cut the bone workpiece 30 according to the requirement of a surgical procedure. In one example, the cut paths 32 are configured to resect or cut a distal or proximal end portion of a bone workpiece 30 for the cooperation of an implant with the rested bone 30, such for a total knee arthroplasty (TKA) that involves resecting and replacing the articular surfaces of a tibia and/or femur (e.g., femoral condyles and tibial plateau).

In some embodiments, the robotic systems 1A, 1B and related methods include an electronic controller that comprises plurality of control inputs that a user can selectively actuate to control the movement of the cutting tool 16 while the robotic systems 1A, 1B is executing one of the determined, prescribed or programmed cut paths 32. In some embodiments, the plurality of control inputs comprise a plurality of manually engageable, activatable or actuatable electrical switches, sensors or the like configured to send an electronic signal or otherwise communicate with the robotic system 1A, 1B to provide the robotic system 1A, 1B with an action or instruction that alters or effects current cutting parameters of the robotic system 1A, 1B. For example, the plurality of control inputs may comprise buttons, paddles, triggers, directional pads, thumbsticks, or the like, and related associated electrical circuitry and componentry.

In some such embodiments, the plurality of control inputs comprises at least one first control input configured such that, when selectively actuated by a user, the robotic system 1A, 1B is directed to advance the cutting tool 16 along a current programmed cut path 32 that the robotic system 1A, 1B is executing. In some such embodiments, the plurality of control inputs comprises at least one second control input configured such that, when selectively actuated by a user, the robotic system 1A, 1B is directed to retreat or reverse the cutting tool 16 along a current programmed cut path 32 that the robotic system 1A, 1B executed immediately previous thereto. In some embodiments, the plurality of control inputs comprises at least one third control input configured such that, when selectively actuated by a user, the robotic system 1A, 1B is directed to translate the cutting tool 16 along a current programmed cut path 32 that the robotic system 1A, 1B is executing at an increased velocity or speed as compared to a current programmed (and effectuated) velocity thereof. In some embodiments, the plurality of control inputs comprises at least one fourth control input configured such that, when selectively actuated by a user, the robotic system 1A, 1B is directed to translate the cutting tool 16 along a current programmed cut path 32 that the robotic system 1A, 1B is executing at a decreased velocity or speed as compared to a current programmed (and effectuated) velocity thereof. In some embodiments, the plurality of control inputs comprises at least one fifth control input configured such that, when selectively actuated by a user, the robotic system 1A, 1B is directed to cease translating the cutting tool 16 along its cutting pathway 17. For example, the at least one fifth control input may shut of the end effector 14 or otherwise cause the end effector 14 to stop powering or translating the cutting tool 16 along its cutting pathway 17. In some embodiments, the plurality of control inputs comprises at least one sixth control input configured such that, when selectively actuated by a user, the robotic system 1A, 1B is directed to register a current point or location of the cutting tool 16 along the cutting pathway 32 that the robotic system 1A, 1B is currently executing.

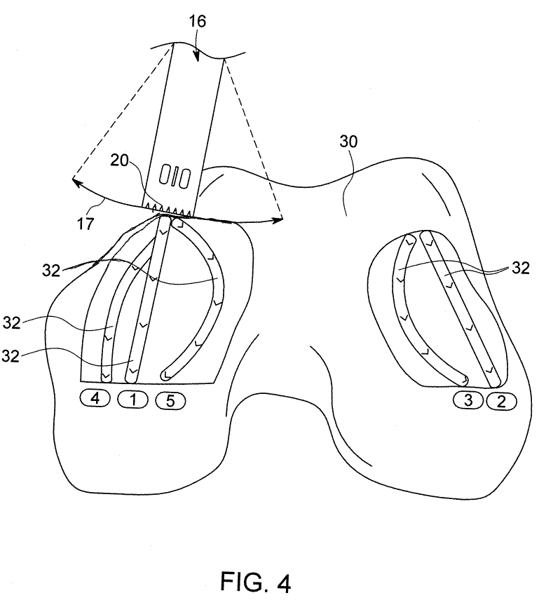

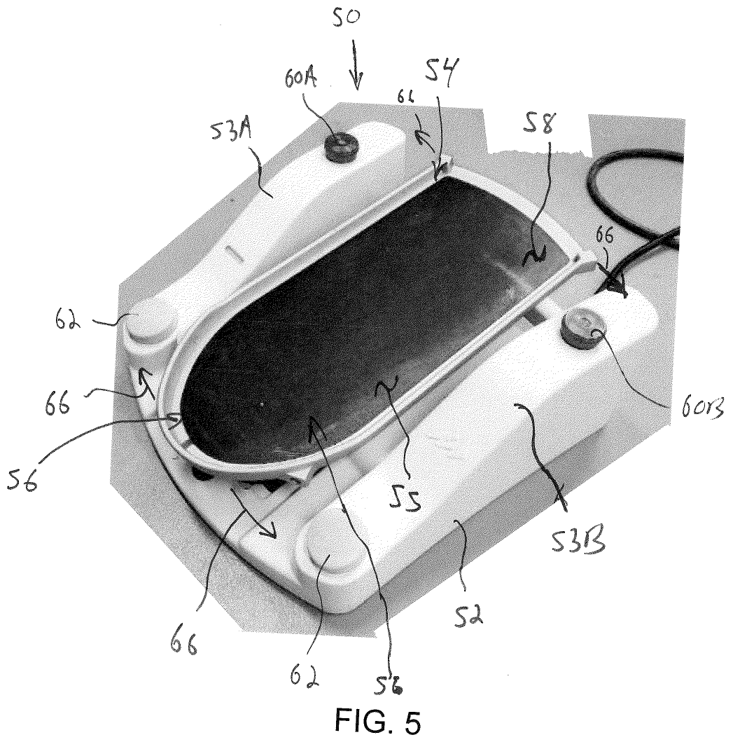

As shown in FIGS. 5-7, in some exemplary embodiments, the robotic system 1A, 1B and related methods may comprise or make use of an exemplary controller 50 that is configured as a foot operated controller such that the plurality of control inputs thereof are configured to be selectively actuated by a user's foot. In some such embodiments, as shown in FIGS. 5-7, the foot controller 50 may comprise a foot pedal 54 that comprises an engagement side or surface 55 that is configured to be selectively engaged by the underside of a user's foot, and a housing that movably supports the foot pedal 54, as shown in FIGS. 5-7. The housing may comprise a base portion 52 that is configured to support the controller 50 and engage a ground surface, and one or more side or peripheral portions 53A, 53B that are positioned adjacent (e.g., medially, laterally, proximally and/or distal) to the foot pedal 54, as shown in FIGS. 5-7.

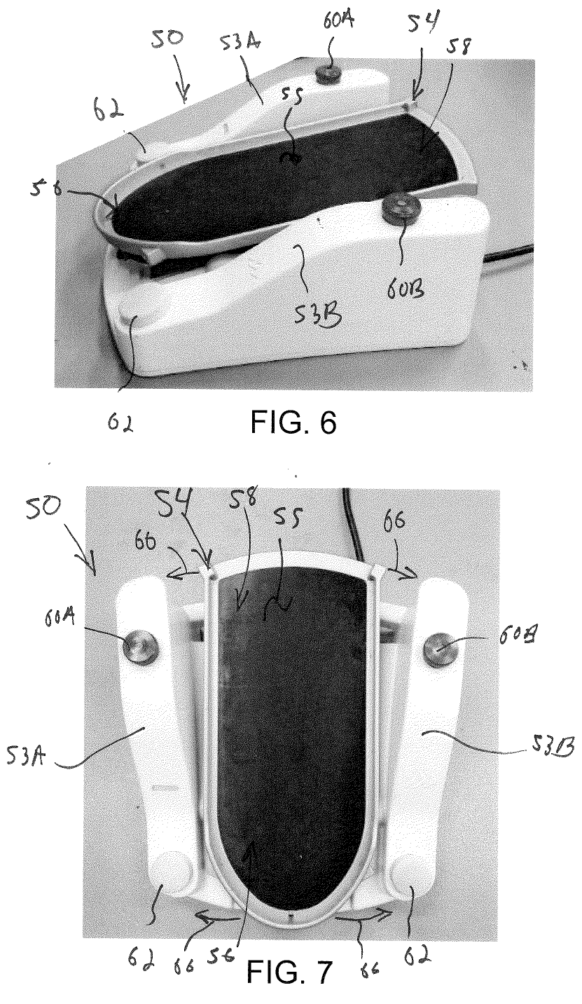

The foot pedal 54 may be pivotably or articulably coupled to the housing. The foot pedal 54 may be configured such that a user can articulate the foot pedal 54 between a first forefoot position with a forefoot portion 58 of the foot pedal 54 being depressed or positioned in a depressed or lowered location as compared to a hindfoot portion 56 of the foot pedal 54 (not shown), and a second hindfoot position with the hindfoot portion 56 of the foot pedal 54 being depressed or positioned in a depressed or lowered location as compared to the forefoot portion 58 of the foot pedal 54 as shown in FIGS. 5 and 6. The forefoot portion 58 of the foot pedal 54 is configured to be positioned beneath and engage a forefoot portion of a user's foot, and the hindfoot portion 56 of the foot pedal 54 is configured to be positioned beneath and engage a hindfoot portion of the user's foot.

In some embodiments, the first forefoot position of the foot pedal 54 actuates a first control input that is configured to direct or control the robotic system 1A, 1B to advance the cutting tool 16 along a current programmed cut path 32 that the robotic system 1A, 1B is executing when the first control input is selectively actuated by a user (via the first forefoot position of the foot pedal 54). In some embodiments, the second hindfoot position of the foot pedal 54 actuates a second control input that is configured to direct or control the robotic system 1A, 1B to retreat or reverse the translation of the cutting tool 16 along a current programmed cut path 32 that the robotic system 1A, 1B executed immediately previous thereto when the second control input is selectively actuated by the user (via the second hindfoot position of the foot pedal 54), as shown in FIGS. 5 and 6.

In some embodiments, the foot pedal 54 is configured such that when positioned in a neutral position between the first forefoot position and the second hindfoot position, the controller 50 directs the robotic system 1A, 1B to stop or pause the cutting tool 16 in its current position along a current programmed cut path 32. In some embodiments, s shown in FIGS. 5 and 7, the foot pedal 54 may be configured to be selectively rotated along a medial-lateral direction 66 by a user's foot. The controller 50 may be configure such that medial-lateral rotation of the foot pedal 54 activates at least one sixth control input that, when selectively actuated by a user via rotation of the foot pedal 54, directs the robotic system 1A, 1B to register a current point of the cutting tool 16 along the cutting pathway 32 that the robotic system 1A, 1B is executing.

As shown in FIGS. 5-7, the housing of the foot controller 50 may comprise a plurality of the plurality of control inputs 60A, 60B, 62 that are configured to be selectively engaged by the user's foot. For example, in some embodiments, a first portion 53A of the housing may comprises a third control input 60A that, when selectively actuated by a user's foot, is configured to direct the robotic system 1A, 1B to translate the cutting tool 16 along a current programmed cut path 32 that the robotic system 1A, 1B is executing at an increased speed or velocity as compared to a current programmed velocity, as shown in FIGS. 5-7. Similarly, in some embodiments, a second portion 53B of the housing may comprises a fourth control input 60B that, when selectively actuated by a user's foot, is configured to direct the robotic system 1A, 1B to translate the cutting tool 16 along a current programmed cut path 32 that the robotic system 1A, 1B is executing at a decreased speed or velocity as compared to a current programmed velocity, as shown in FIGS. 5-7. In some embodiments, the first and second portions 53B of the housing may be on opposing sides of the foot pedal 54, such as positioned on medial and lateral sides thereof.

In some embodiments, the housing may comprise a fourth control input 62 that, when selectively actuated by a user's foot, is configured to direct the robotic system 1A, 1B to stop or pause the end effector 14 from translating the cutting tool 16 along its cutting pathway 17. In some such embodiments, the fourth control input 62 may also be configured such that when selectively actuated by a user's foot, the robotic system 1A, 1B is directed to stop or pause the cutting tool 16 in its current position along a current programmed cut path 32.

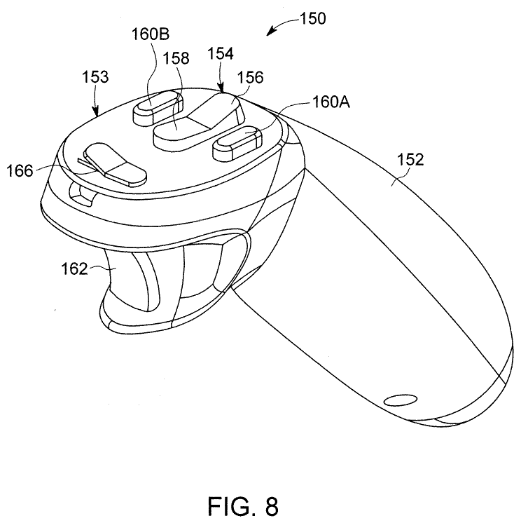

As shown in FIG. 8, in some exemplary embodiments, the robotic system 1A, 1B and related methods may comprise or make use of an exemplary controller 150 that is configured as a hand operated controller. The controller 150 may be configured as a handheld controller such that the plurality of control inputs are configured to be selectively actuated by one or more fingers of a hand of the user, as shown in FIG. 8.

In some embodiments, the handheld controller 150 may comprises a grip portion 152 configured to be grasped by a user's palm and one more fingers of the user's hand, and a head portion 153 comprising a plurality of the plurality of control inputs on an upper side thereof, as shown in FIG. 8. For example, as shown in FIG. 8, in some embodiment, the head portion 153 of the handheld controller 150 may include at least one first control input 158 that, when selectively actuated by a user (e.g., via a user's thumb), is configured to direct the robotic system 1A, 1B to advance the cutting tool 16 along a current programmed cut path 32 that the robotic system 1A, 1B is executing. In some embodiment, the head portion 153 of the handheld controller 150 may include at least one second control input 156 that, when selectively actuated by a user, is configured to direct the robotic system 1A, 1B to retreat or reverse the translation of the cutting tool 16 along a current programmed cut path 32 that the robotic system 1A, 1B executed immediately previous thereto when the second control input 156 is selectively actuated by the user (e.g., via a user's thumb). In some embodiments, as shown in FIG. 8, the first control input 158 and the second control input 156 may be portions of a rocker switch, direction pad, stick or other multi-portion control input device 154.

In some embodiment, as shown in FIG. 8, the head portion 153 of the handheld controller 150 may include at least one third control input 160A that, when selectively actuated by a user (e.g., via a user's thumb), is configured to direct the robotic system 1A, 1B to translate the cutting tool along a current programmed cut path 32 that the robotic system 1A, 1B is executing at an increased velocity as compared to a current programmed velocity. Similarly, as shown in FIG. 8, the head portion 153 of the handheld controller 150 may include at least one fourth control input 160B that, when selectively actuated by a user (e.g., via a user's thumb), is configured to direct the robotic system 1A, 1B to translate the cutting tool along a current programmed cut path 32 that the robotic system 1A, 1B is executing at a decreased velocity as compared to a current programmed velocity.

In some embodiments, as shown in FIG. 8, the head portion 153 of the handheld controller 150 may include at least one fifth control input 166 that, when selectively actuated by a user (e.g., via a user's thumb), is configured to direct the robotic system 1A, 1B to register a current point of the cutting tool 16 along the cutting pathway 32 that the robotic system 1A, 1B is executing. As also shown in FIG. 8, in some embodiments a front portion of the head portion 153 may comprise at least one sixth control input 162 that, when selectively actuated by a user, is configured to direct the robotic system 1A, 1B to stop or pause the end effector 14 from translating the cutting tool 16 along its cutting pathway 17. In some such embodiments, the sixth control input 162 may also be configured such that when selectively actuated by a user, the robotic system 1A, 1B is directed to stop or pause the cutting tool 16 in its current position along a current programmed cut path 32. In some embodiments, the sixth control input 162 may be configured to be selectively actuated by a user's index and/or middle finger.

It is also noted that the cut paths described herein represent the general or overall spatial pathway of the cutting edge of the cutting tool/blade 16 as it is translated via a active/autonomous robotic system 1A, 1B to and through the respective workpiece/bone portion (i.e., during a cutting operation). A cut path may be formed or completed via a single continuous cutting motion or advancement of the cutting tool/blade 16 directly along the respective pathway. A cut path may also be formed or completed via a plurality of consecutive separate and distinct cutting motions or advancements of the cutting tool/blade 16 that cooperatively extend along the respective pathway. For example, a system 1A, 1B can be programmed to perform a series of small advancements or intermediate cutting paths (which may or may not be sequential) that each remove small amounts of workpiece/bone along a given vector (i.e., peck at the bone). The cutting tool/blade 10 may stop, move backwards or otherwise translate off of the cut path between the small advancements or intermediate cutting paths, but the collective workpiece/bone removal of the series of small advancements or intermediate cutting paths may form the respective cut path. A cut path, as used herein, is thus the overall pathway, trajectory or strip of removed workpiece/bone portions during a cutting operation. The actual movement of the cutting tool/blade 16 that forms the cut path may or may not extend directly along the cut path. Accordingly, a cut path disclosed herein does not require that the cutting blade 16 move continuously along a given path or trajectory, but rather represents a path or strip of removed workpiece/bone without reference to exactly how the cutting blade 10 exactly cuts/removes the workpiece/bone portion, but rather is the end result of an overall cutting operation. It is also noted that cut paths may or may not overlap with each other.

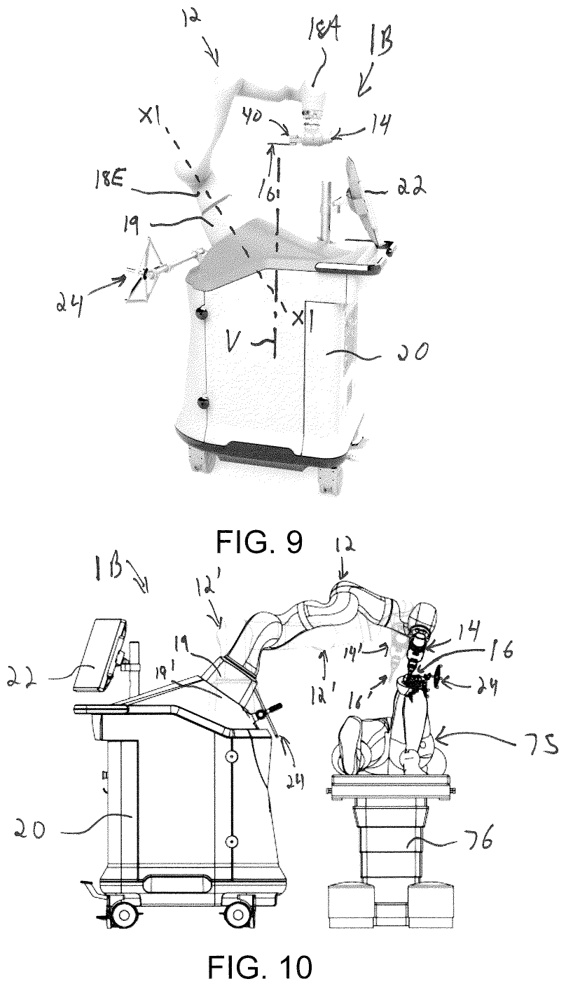

As discussed above, and as shown in the exemplary robotic system 1B illustrated in FIGS. 9 and 10, the base portion 10 and the first arm segment 18E of the articulated/articulating arm 12 may be angled with respect to vertical such that the base portion 10 and the first arm segment 18E extend away or out from a respective side/end of the cart or base 20 (e.g., a forward side, section or end) as they extend upwardly therefrom. It is also noted that the base portion 10 and the first arm segment 18E are mounted proximate to the respective side/end of the cart or base 20 (e.g., a forward side, section or send), and the arm 12 is thereby cantilevered over such respective side/end of the cart or base 20. For example, in some embodiments, the base portion 10 and/or the first arm segment 18E may define an axis X1-X1 that is angled within the range of about 15 degrees to about 55 degrees from vertical V, or within the range of about 25 degrees to about 25 degrees from vertical V. In the exemplary embodiment illustrated in FIGS. 9 and 10, the axis X1-X1 of the base portion 10 and/or the first arm segment 18E is angled about 35 degrees from vertical V.

It is noted that robotic systems are generally constrained by their physical limits. Generally speaking, it may be preferable to position the articulating/articulated arm in a work space such that there is kinematic redundancy to the execution of the required task. Furthermore, it is generally preferable to maximize the manipulability of the arm within a workspace such that the required movement of the joints is minimized for performing certain tasks (i.e., such that the tasks can be executed in a time efficient manner). The position of the arm on the cart or base 02 relative to site of interest of the workpiece 75 is critical to the optimal performance of the system. By way of a nonlimiting example, unoptimized positioning of the arm on the cart or base 20, and/or the cart or base 20 relative to the target site of the workpiece 75, could require repositioning of the cart or base 20 during a cutting operation or procedure, or could require repositioning of the workpiece/patient 75 during the cutting operation/procedure, or could require changing tools to execute all desired cuts.

The angled base portion 10 and the first arm segment 18E of the articulating/articulated arm 12 unexpectedly maximizes the working area around the workpiece (e.g., a patient or other workpiece 75 on a surgical table or support structure 76) without compromising the working envelope of the robot. For example, as shown in FIG. 10, the cart or base 20 of a robotic system 1B with an articulating/articulated robotic arm 12′ that includes a vertically mounted/oriented base portion 10′ and first arm segment 18E′ would need to be positioned closer to the workpiece/patient 75, and/or the arm 12′ would not have the same reach, as compared to the articulating/articulated arm 12 with the angled base portion 10 and the first arm segment 18E. It is noted that if the angled base portion 10 and the first arm segment 18E were positioned horizontally, the arm 12 would be closer to the floor and potentially below the workpiece/patient 75, and a degree-of-freedom of the arm 12 would be eliminated. The angled arm 12 with the cantilever/angled base portion 10 and the first arm segment 18E thereby maximizes the working volume of the arm 12 and the robotic system 1B.

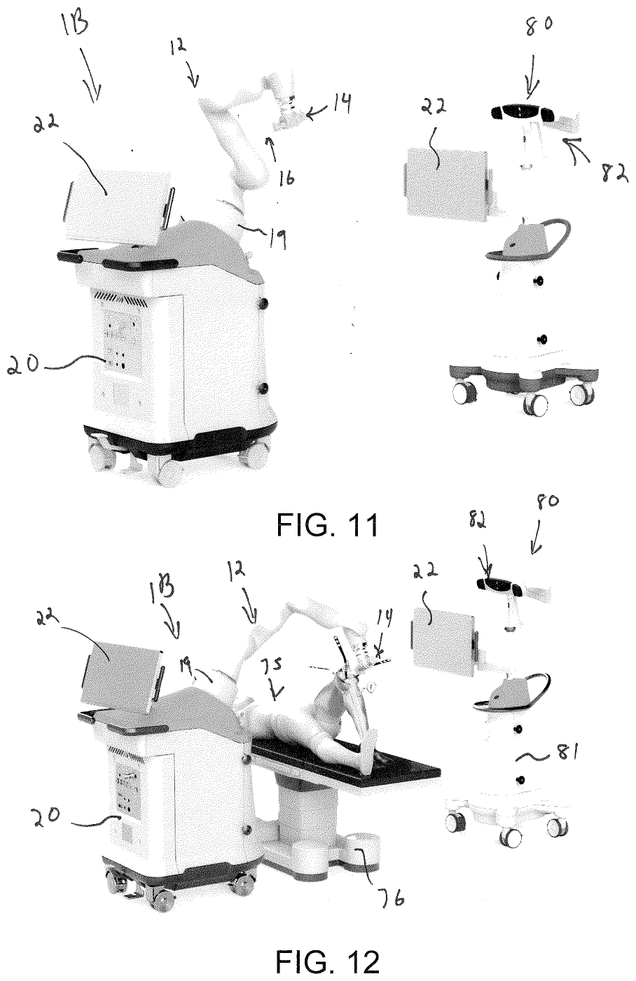

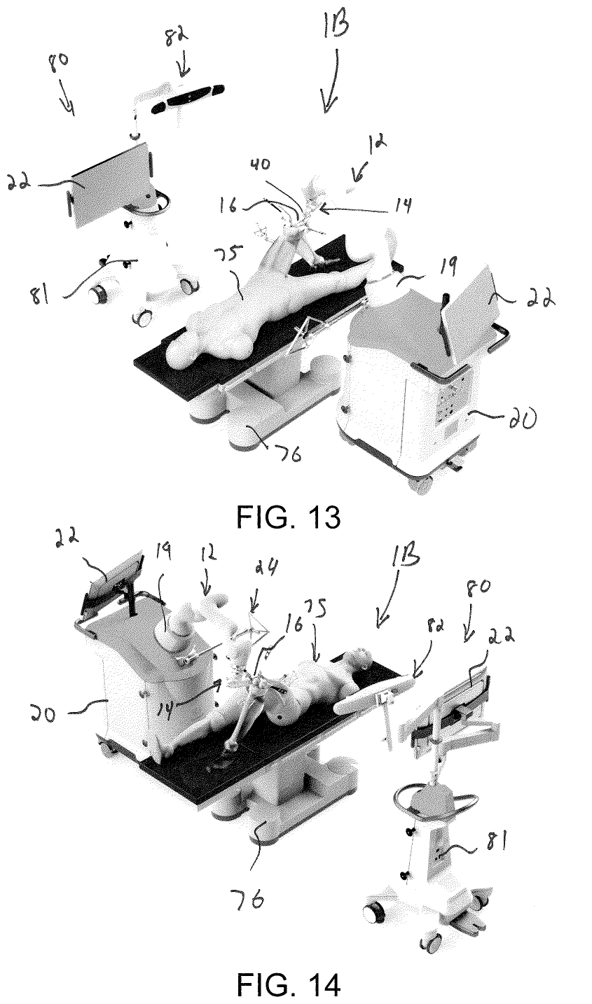

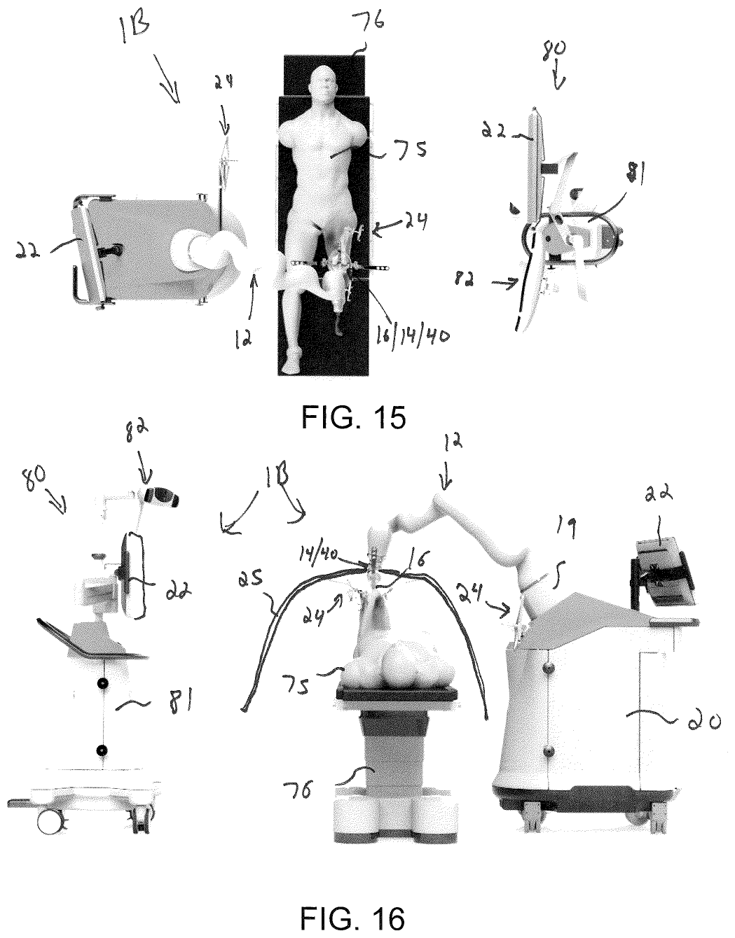

With reference to FIGS. 11-16, a robotic system including the cart-mounted autonomous/active surgical cutting robotic system 1B and a separate and distinct navigation or guidance cart 80 that communicates with and assists the operation of the cutting robotic system 1B is illustrated.

It is noted that certain robotic applications may necessitate the use of carts for robotic system mobility. Certain applications may require a robotic system that supports tracking cameras, monitors, or other equipment. Furthermore, the robotic systems 1A, 1B may require a user input station(s) that facilitate inputs to the system by an operator or other user(s). The optimal positioning and configuration of such user interfaces is not intuitive and can have safety, accuracy and other performance implications.

As shown in FIGS. 11-16, a robotic system according to the present disclosure may an active/autonomous cutting robot mounted on a robot cart 20 with at least one display or monitor 22, a computing system, and a navigation or guidance cart 80 that includes at least one tracking camera 82 and at least one display or monitor 22. The robot cart 20 and the guidance cart 80 may each include lockable wheels that allow for selective movement or translation thereof, respectively, as shown in FIGS. 11-16.

The at least one tracking camera 82 of the guidance cart 80 may be effective in imaging the cutting tool 16 and the cart 20, and potentially the patient/workpiece 75, so that the relative three-dimensional positions and orientations thereof can be determined/tracked to, ultimately, determine the necessary adjustments of the arm 12 to autonomously effectuate one or more cut paths via the cutting tool 16. In such embodiments, the robot cart 20 may be void of any such tracking cameras or the like 82. It is noted that movement of any of the tracking cameras 82 of the robot system could materially diminish tracking accuracy. For example, at a distance of 2 meters of an object, a 1 degree movement of a camera 82 could introduce over 34 mm of tracking error of the object. The robot cart 20 may include the tracking array 24 for localizing the robot arm base 19 and/or base segment 18E (for example) via the tracking camera system 82 and monitor 22 of the guidance cart 80.

The guidance cart 80, supporting the at least one camera system 82 and at least one screen/monitor 22, may be configured sch that it can be manually positioned and then locked generally in place via a base 81 that includes wheels/casters. The at least one camera system 82 and/or the at least one screen/monitor 22 may be positioned via movable mounts that are semi-rigid self-stabilized or locking after positioning thereof, for example. In some embodiments, the at least one screen/monitor 22 of the guidance cart 80 may be passive such that it is configured to not operate as a tough screen or other user input device.

As also shown in FIGS. 11-16 and described above, the rolling robot cart 20 may include the robotic arm 12 and cutting components (e.g., end effector 14, attachment mechanism 40 and cutting tool 16, for example) positioned at or towards a forward section of the cart 20 and angled towards its closest edge. The angle of the robot arm base 19 and/or base segment 18E may be between 0 degrees and 90 degrees from vertical. In some embodiments, the robot arm base 19 and/or base segment 18E may angled within the range of about 15 degrees to about 55 degrees from vertical, or within the range of about 25 degrees to about 25 degrees from vertical, or be angled about 35 degrees from vertical. As shown in FIGS. 11-16, the cutting robot cart 20 may also include at least one monitor/screen 22, and potentially a workstation, positioned towards the posterior portion of the cart 22 opposite or spaced from the robot arm base 19 and/or base segment 18E. In some embodiments, the at least one monitor/screen 22 may be mounted such that it can articulate around a rigid post and such that its height can be variable, for example.

In some embodiments, at shown in FIGS. 11-16, the cutting robot cart 20 may include a flat work surface configured for/with a keyboard, and may include retracting storage drawers (for storage of a keyboard and mouse or other input device, for example). The posterior section of the cart 20 may be configured for inputs from a primary operator or user. In some embodiments, the screen/monitor 22 mounted on the cart 20 may be a touchscreen. In some embodiments, the cart 20 may house a main control and computing system, auxiliary power unit and/or other miscellaneous electronics.

It is noted that the robotic system with the cart-mounted robotic cutting system 1B and the navigation/guidance cart 80 descried above advantageously minimizes user interaction with the at least one tracking camera system 82 (which can introduce tracking error), and maximize the working area around the patient/workpiece 75 (and support/surgical table 76) without compromising the working envelope of the cutting robot.

It is to be understood that the above description is intended to be illustrative, and not restrictive. For example, the above-described exemplary embodiments, and/or aspects thereof, may be used in combination with each other. In addition, many modifications may be made to adapt a particular configuration according to the teachings of the various examples without departing from their scope. For example, it is expressly disclosed that the cutting tool 16 may not be configured as a sagittal saw blade or a rotary cutter, but rather a differing type of cutting tool. Further, the specific configuration and operation of the plurality of control inputs may be altered to differing user actuated input devices and/or configurations with departing from the spirit and scope of the present disclosure. For example, the controllers disclosed herein may include additional control inputs, fewer f control inputs or differing styles and/or functioning control inputs. Still further, the plurality of control inputs may be assigned to different control parameters of the movement of the cutting tool along a determined, prescribed or programmed cut path.

Processes described herein may be performed singly or collectively by one or more computer systems, such as one or more systems that are, or are in communication with, the robotic system, such as the articulating joints, end effector, controller, camera system, tracking system, and/or AR system thereof, as examples. FIG. 17 depicts one example of such a computer system and associated devices to incorporate and/or use aspects described herein. A computer system may also be referred to herein as a data processing device/system, computing device/system/node, or simply a computer. The computer system may be based on one or more of various system architectures and/or instruction set architectures, such as those offered by Intel Corporation (Santa Clara, California, USA) or ARM Holdings plc (Cambridge, England, United Kingdom), as examples.

FIG. 17 shows a computer system 200 in communication with external device(s) 212. Computer system 200 includes one or more processor(s) 202, for instance central processing unit(s) (CPUs). A processor can include functional components used in the execution of instructions, such as functional components to fetch program instructions from locations such as cache or main memory, decode program instructions, and execute program instructions, access memory for instruction execution, and write results of the executed instructions. A processor 202 can also include register(s) to be used by one or more of the functional components. Computer system 200 also includes memory 204, input/output (I/O) devices 208, and I/O interfaces 210, which may be coupled to processor(s) 202 and each other via one or more buses and/or other connections. Bus connections represent one or more of any of several types of bus structures, including a memory bus or memory controller, a peripheral bus, an accelerated graphics port, and a processor or local bus using any of a variety of bus architectures. By way of example, and not limitation, such architectures include the Industry Standard Architecture (ISA), the Micro Channel Architecture (MCA), the Enhanced ISA (EISA), the Video Electronics Standards Association (VESA) local bus, and the Peripheral Component Interconnect (PCI).

Memory 204 can be or include main or system memory (e.g., Random Access Memory) used in the execution of program instructions, storage device(s) such as hard drive(s), flash media, or optical media as examples, and/or cache memory, as examples. Memory 204 can include, for instance, a cache, such as a shared cache, which may be coupled to local caches (examples include L1 cache, L2 cache, etc.) of processor(s) 202. Additionally, memory 204 may be or include at least one computer program product having a set (e.g., at least one) of program modules, instructions, code or the like that is/are configured to carry out functions of embodiments described herein when executed by one or more processors.

Memory 204 can store an operating system 205 and other computer programs 206, such as one or more computer programs/applications that execute to perform aspects described herein. Specifically, programs/applications can include computer readable program instructions that may be configured to carry out functions of embodiments of aspects described herein.

Examples of I/O devices 208 include but are not limited to microphones, speakers, Global Positioning System (GPS) devices, RGB and/or IR cameras, lights, accelerometers, gyroscopes, magnetometers, sensor devices configured to sense light, proximity, heart rate, body and/or ambient temperature, blood pressure, and/or skin resistance, registration probes and activity monitors. An I/O device may be incorporated into the computer system as shown, though in some embodiments an I/O device may be regarded as an external device (212) coupled to the computer system through one or more I/O interfaces 210.

Computer system 200 may communicate with one or more external devices 212 via one or more I/O interfaces 210. Example external devices include a keyboard, a pointing device, a display, and/or any other devices that enable a user to interact with computer system 200. Other example external devices include any device that enables computer system 200 to communicate with one or more other computing systems or peripheral devices such as a printer. A network interface/adapter is an example I/O interface that enables computer system 200 to communicate with one or more networks, such as a local area network (LAN), a general wide area network (WAN), and/or a public network (e.g., the Internet), providing communication with other computing devices or systems, storage devices, or the like. Ethernet-based (such as Wi-Fi) interfaces and Bluetooth® adapters are just examples of the currently available types of network adapters used in computer systems (BLUETOOTH is a registered trademark of Bluetooth SIG, Inc., Kirkland, Washington, U.S.A.).

The communication between I/O interfaces 210 and external devices 212 can occur across wired and/or wireless communications link(s) 211, such as Ethernet-based wired or wireless connections. Example wireless connections include cellular, Wi-Fi, Bluetooth®, proximity-based, near-field, or other types of wireless connections. More generally, communications link(s) 211 may be any appropriate wireless and/or wired communication link(s) for communicating data.

Particular external device(s) 212 may include one or more data storage devices, which may store one or more programs, one or more computer readable program instructions, and/or data, etc. Computer system 200 may include and/or be coupled to and in communication with (e.g., as an external device of the computer system) removable/non-removable, volatile/non-volatile computer system storage media. For example, it may include and/or be coupled to a non-removable, non-volatile magnetic media (typically called a “hard drive”), a magnetic disk drive for reading from and writing to a removable, non-volatile magnetic disk (e.g., a “floppy disk”), and/or an optical disk drive for reading from or writing to a removable, non-volatile optical disk, such as a CD-ROM, DVD-ROM or other optical media.

Computer system 200 may be operational with numerous other general purpose or special purpose computing system environments or configurations. Computer system 200 may take any of various forms, well-known examples of which include, but are not limited to, personal computer (PC) system(s), server computer system(s), such as messaging server(s), thin client(s), thick client(s), workstation(s), laptop(s), handheld device(s), mobile device(s)/computer(s) such as smartphone(s), tablet(s), and wearable device(s), multiprocessor system(s), microprocessor-based system(s), telephony device(s), network appliance(s) (such as edge appliance(s)), virtualization device(s), storage controller(s), set top box(es), programmable consumer electronic(s), network PC(s), minicomputer system(s), mainframe computer system(s), and distributed cloud computing environment(s) that include any of the above systems or devices, and the like.

Various input devices may be provided, such as a camera, which can be used to capture images or video. The camera can be used by the device to obtain image(s)/video of a view of the material to be cut and/or the cutting tool, for instance, capturing images/videos of a scene.

One or more microphones, proximity sensors, light sensors, accelerometers, speakers, GPS devices, and/or other input devices (not labeled) may be additionally provided. Electronic components, such as electronic circuitry, including processor(s), memory, and/or communications devices, such as cellular, short-range wireless (e.g., Bluetooth), or Wi-Fi circuitry for connection to remote devices may be included. A power source, such as a battery to power components of the system may also be incorporated. Physical port(s) (not pictured) used to connect device the computer to a power source (to recharge a battery) and/or any other external device, such as the controller. Such physical ports can be of any standardized or proprietary type, such as Universal Serial Bus (USB).

Aspects of the present invention may be a system, a method, and/or a computer program product, any of which may be configured to perform or facilitate aspects described herein.

In some embodiments, aspects of the present invention may take the form of a computer program product, which may be embodied as computer readable medium(s). A computer readable medium may be a tangible storage device/medium having computer readable program code/instructions stored thereon. Example computer readable medium(s) include, but are not limited to, electronic, magnetic, optical, or semiconductor storage devices or systems, or any combination of the foregoing. Example embodiments of a computer readable medium include a hard drive or other mass-storage device, an electrical connection having wires, random access memory (RAM), read-only memory (ROM), erasable-programmable read-only memory such as EPROM or flash memory, an optical fiber, a portable computer disk/diskette, such as a compact disc read-only memory (CD-ROM) or Digital Versatile Disc (DVD), an optical storage device, a magnetic storage device, or any combination of the foregoing. The computer readable medium may be readable by a processor, processing unit, or the like, to obtain data (e.g., instructions) from the medium for execution. In a particular example, a computer program product is or includes one or more computer readable media that includes/stores computer readable program code to provide and facilitate one or more aspects described herein.

As noted, program instruction contained or stored in/on a computer readable medium can be obtained and executed by any of various suitable components such as a processor of a computer system to cause the computer system to behave and function in a particular manner. Such program instructions for carrying out operations to perform, achieve, or facilitate aspects described herein may be written in, or compiled from code written in, any desired programming language. In some embodiments, such programming language includes object-oriented and/or procedural programming languages such as C, C++, C#, Java, etc.

Program code can include one or more program instructions obtained for execution by one or more processors. Computer program instructions may be provided to one or more processors of, e.g., one or more computer systems, to produce a machine, such that the program instructions, when executed by the one or more processors, perform, achieve, or facilitate aspects of the present invention, such as actions or functions described in flowcharts and/or block diagrams described herein. Thus, each block, or combinations of blocks, of the flowchart illustrations and/or block diagrams depicted and described herein can be implemented, in some embodiments, by computer program instructions.

Many other examples will be apparent to those of skill in the art upon reviewing the above description. The scope of the various examples should, therefore, be determined with reference to the appended claims, along with the full scope of equivalents to which such claims are entitled.

While dimensions and types of materials may be described herein, they are intended to define parameters of some of the various examples, and they are by no means limiting to all examples and are merely exemplary.

In the appended claims, the terms “including” and “in which” are used as the plain-English equivalents of the respective terms “comprising” and “wherein.” Moreover, in the following claims, the terms “first,” “second,” and “third,” etc. are used merely as referee labels, and are not intended to impose numerical, structural or other requirements on their objects. Forms of term “based on” herein encompass relationships where an element is partially based on as well as relationships where an element is entirely based on. Forms of the term “defined” encompass relationships where an element is partially defined as well as relationships where an element is entirely defined. Further, the limitations of the following claims are not written in means-plus-function format and are not intended to be interpreted based on 35 U.S.C. § 112, sixth paragraph, unless and until such claim limitations expressly use the phrase “means for” followed by a statement of function cavity of further structure. It is to be understood that not necessarily all such objects or advantages described above may be achieved in accordance with any particular example. Thus, for example, those skilled in the art will recognize that the devices, systems and methods described herein may be embodied or carried out in a manner that achieves or optimizes one advantage or group of advantages as taught herein without necessarily achieving other objects or advantages as may be taught or suggested herein.

While the disclosure has been described in detail in connection with only a limited number of examples, it should be readily understood that the disclosure is not limited to such disclosed examples. Rather, this disclosure can be modified to incorporate any number of variations, alterations, substitutions or equivalent arrangements not heretofore described, but which are commensurate with the spirit and scope of the disclosure. Additionally, while various examples have been described, it is to be understood that aspects of the disclosure may include only one example or some of the described examples. Also, while some disclosure are described as having a certain number of elements, it will be understood that the examples can be practiced with less than or greater than the certain number of elements.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

We claim:

1. A robotic system comprising:
an articulated arm comprising a plurality of arm segments defining longitudinal axes, and adjustable joints coupled between adjacent arm segments that are configured to adjust the orientation of the axes of the adjacent arm segments;
a cutting tool defining a cutting edge that is configured to cut while being translated along a cutting pathway;
an end effector coupled to an end arm segment of the plurality of arm segments and the cutting tool, the end effector comprising a powered drive portion that translates the cutting tool along the cutting pathway; and
an electronic controller,
wherein the robotic system is configured to autonomously adjust the relative orientation of the arm segments of the articulated arm while the cutting tool translates along the cutting pathway to autonomously spatially translate the cutting tool and cut a material along at least one programmed cut path, and
wherein the controller comprises a plurality of control inputs that a user can selectively actuate to control the operation of the cutting tool while executing the at least one programmed cut path but not alter the configuration of the at least one programmed cut path,
wherein the plurality of control inputs comprises a first control input that, when selectively actuated by a user, is configured to direct the robotic system to advance the cutting tool along a current programmed cut path that the robotic system is executing.

2. The robotic system of claim 1, wherein the plurality of control inputs comprises a second control input that, when selectively actuated by a user, is configured to direct the robotic system to retreat the cutting tool along a current programmed cut path that the robotic system executed immediately previous thereto.

3. The robotic system of claim 2, wherein the plurality of control inputs comprises a third control input that, when selectively actuated by a user, is configured to direct the robotic system to translate the cutting tool along a current programmed cut path that the robotic system is executing at an increased velocity as compared to a current programmed velocity.

4. The robotic system of claim 3, wherein the plurality of control inputs comprises a fourth control input that, when selectively actuated by a user, is configured to direct the robotic system to translate the cutting tool along a current programmed cut path that the robotic system is executing at a decreased velocity as compared to a current programmed velocity.

5. The robotic system of claim 4, wherein the plurality of control inputs comprises a fifth control input that, when selectively actuated by a user, is configured to stop the end effector from translating the cutting tool along the cutting pathway.

6. The robotic system of claim 5, wherein the plurality of control inputs comprises a sixth control input that, when selectively actuated by a user, is configured to register a current point of the cutting tool along the cutting pathway that the robotic system is executing.

7. The robotic system of claim 1, wherein the plurality of control inputs comprises a plurality of control inputs selected from a group consisting of:
the first control input;
a second control input that, when selectively actuated by a user, is configured to direct the robotic system to retreat the cutting tool along a current programmed cut path that the robotic system executed immediately previous thereto;
a third control input that, when selectively actuated by a user, is configured to direct the robotic system to translate the cutting tool along a current programmed cut path that the robotic system is executing at an increased velocity as compared to a current programmed velocity;
a fourth control input that, when selectively actuated by a user, is configured to direct the robotic system to translate the cutting tool along a current programmed cut path that the robotic system is executing at a decreased velocity as compared to a current programmed velocity;
a fifth control input that, when selectively actuated by a user, is configured to stop the end effector from translating the cutting tool along the cutting pathway; and
a sixth control input that, when selectively actuated by a user, is configured to register a current point of the cutting tool along the cutting pathway that the robotic system is executing.

8. The robotic system of claim 1, wherein the plurality of control inputs comprises at least four control inputs selected from a group consisting of:
a first control input that, when selectively actuated by a user, is configured to direct the robotic system to advance the cutting tool along a current programmed cut path that the robotic system is executing;
a second control input that, when selectively actuated by a user, is configured to direct the robotic system to retreat the cutting tool along a current programmed cut path that the robotic system executed immediately previous thereto;
a third control input that, when selectively actuated by a user, is configured to direct the robotic system to translate the cutting tool along a current programmed cut path that the robotic system is executing at an increased velocity as compared to a current programmed velocity;
a fourth control input that, when selectively actuated by a user, is configured to direct the robotic system to translate the cutting tool along a current programmed cut path that the robotic system is executing at a decreased velocity as compared to a current programmed velocity;
a fifth control input that, when selectively actuated by a user, is configured to stop the end effector from translating the cutting tool along the cutting pathway; and
a sixth control input that, when selectively actuated by a user, is configured to register a current point of the cutting tool along the cutting pathway that the robotic system is executing.

9. The robotic system of claim 1, wherein the plurality of control inputs comprise a plurality of electrical switches.

10. The robotic system of claim 1, wherein the controller is configured as a foot controller such that the plurality of control inputs are configured to be selectively actuated by a user's foot.

11. The robotic system of claim 10, wherein the foot controller comprises a foot pedal that is configured to be selectively engaged by the underside of a user's foot and a housing positioned adjacent to at least one side of the foot pedal, and wherein the foot pedal is configured such that the user can articulate the foot pedal between a first forefoot position with a forefoot portion of the foot pedal being depressed, and a second hindfoot position with a hindfoot portion of the foot pedal being depressed.

12. The robotic system of claim 11, wherein the first forefoot position of the foot pedal actuates a first control input that is configured to direct the robotic system to advance the cutting tool along a current programmed cut path that the robotic system is executing when selectively actuated by a user.

13. The robotic system of claim 11, wherein the second hindfoot position of the foot pedal actuates a second control input second control input that is configured to direct the robotic system to retreat the cutting tool along a current programmed cut path that the robotic system executed immediately previous thereto when selectively actuated by a user.

14. The robotic system of claim 11, wherein the housing comprises a plurality of the plurality of control inputs that are configured to be selectively engaged by the user's foot.

15. The robotic system of claim 14, wherein the housing comprises a third control input that, when selectively actuated by a user, is configured to direct the robotic system to translate the cutting tool along a current programmed cut path that the robotic system is executing at an increased velocity as compared to a current programmed velocity.

16. The robotic system of claim 14, wherein the housing comprises a fourth control input that, when selectively actuated by a user, is configured to direct the robotic system to translate the cutting tool along a current programmed cut path that the robotic system is executing at a decreased velocity as compared to a current programmed velocity.

17. The robotic system of claim 16, wherein the third and fourth control inputs are positioned on opposing lateral sides of the foot pedal.

18. The robotic system of claim 17, wherein the housing comprises a fifth control input that, when selectively actuated by a user, is configured to stop the end effector from translating the cutting tool along the cutting pathway.

19. The robotic system of claim 17, wherein the foot pedal is configured to be selectively rotated along a medial-lateral direction by a user's foot, and wherein medial-lateral rotation of the foot pedal is configured to activate a sixth control input that, when selectively actuated by a user, is configured to register a current point of the cutting tool along the cutting pathway that the robotic system is executing.

20. The robotic system of claim 19, wherein the controller is configured as a handheld controller such that the plurality of control inputs are configured to be selectively actuated by one or more fingers of a hand of the user.

21. The robotic system of claim 20, wherein the handheld controller comprises a grip portion configured to be grasped by a user's palm and one more fingers of the user's hand, and a head portion comprising a plurality of the plurality of control inputs on an upper side thereof.

22. The robotic system of claim 21, wherein the plurality of control inputs on the head portion comprise at least one of:
the first control input;
a second control input that, when selectively actuated by a user, is configured to direct the robotic system to retreat the cutting tool along a current programmed cut path that the robotic system executed immediately previous thereto;
a third control input that, when selectively actuated by a user, is configured to direct the robotic system to translate the cutting tool along a current programmed cut path that the robotic system is executing at an increased velocity as compared to a current programmed velocity;
a fourth control input that, when selectively actuated by a user, is configured to direct the robotic system to translate the cutting tool along a current programmed cut path that the robotic system is executing at a decreased velocity as compared to a current programmed velocity; and
a fifth control input that, when selectively actuated by a user, is configured to register a current point of the cutting tool along the cutting pathway that the robotic system is executing.

23. The robotic system of claim 1, wherein the cutting tool is a rotary cutting tool, and wherein the rotary cutting tool is configured to cut when translated longitudinally and rotated about a longitudinal an axis thereof.

24. The robotic system of claim 1, wherein the cutting tool is a sagittal cutting blade with cutting teeth positioned at a longitudinal end thereof, the sagittal cutting blade being configured to cut when translated longitudinally and oscillated along the cutting pathway that lies in a plane in which the blade is aligned about an axis of oscillation.

25. The robotic system of claim 1, wherein the cutting tool is configured to cut bone, and wherein the at least one programmed cut path extends through a first bone of a patient such that the cutting tool cuts the first bone when executing the least one programmed cut path.

26. The robotic system of claim 1, wherein the robotic system is operated by a computer system.

27. The robotic system of claim 1, wherein the robotic system further comprises a selectively movable cutting robot cart, and wherein a base arm segment of the plurality of arm segments of the articulated arm is coupled to the cutting robot cart via a rotatable base portion of the articulated arm.

28. The robotic system of claim 27, wherein the robotic system further comprises at least one of a monitor and a computer input device mounted on a first side portion of the cutting robot cart, and the base portion of the articulated arm is mounted on a second side portion of the cutting robot cart.

29. The robotic system of claim 27, wherein the base arm segment and the base portion are mounted on the cutting robot cart at an angle with respect to vertical.

30. The robotic system of claim 29, wherein the base arm segment and the base portion define axis, and wherein their axis are angled with respect to vertical within the range of 15 degrees and 55 degrees.

31. The robotic system of claim 29, wherein the base arm segment and the base portion define axis, and wherein their axis are angled with respect to vertical at about 35 degrees.

32. The robotic system of claim 27, further comprising a selectively movable guidance cart that comprises a tracking camera system.

33. The robotic system of claim 32, wherein the cutting robot cart further comprises a tracking array, and wherein the system is configured to track and/or localize the articulated arm via the tracking camera system of the guidance cart.

34. The robotic system of claim 32, wherein the guidance cart further comprises at least one monitor that is not configured as a touch screen.

35. The robotic system of claim 1, wherein a connector assembly coupled the articulated arm and the end effector, wherein the connector assembly includes a flange portion, and wherein the flange portion includes a surgical drape coupled thereto that forms a sterility barrier between the articulated arm and the end effector.

36. A method of cutting a material, comprising:
utilizing the robotic system according to claim 1 to translate the cutting tool along the cutting pathway and the least one programmed cut path to cut the material.

37. The method of claim 36, wherein the material comprises a bone of a mammalian patient, and wherein the cutting tool comprises a rotary cutting tool or a sagittal cutting blade.
